(19) United States
(12) Patent Application Publication
(10) Pub. No.: US 2022/0397566 A1
(43) Pub. Date: Dec. 15, 2022

(54) GAMMA-SECRETASE STABILIZING COMPOUND SCREENING ASSAY

(71) Applicants: VIB VZW, Gent (BE); KATHOLIEKE UNIVERSITEIT LEUVEN, K.U.LEUVEN R&D, Leuven (BE)

(72) Inventors: Wim Annaert, Kontich (BE); Ragna Sannerud, Leuven (BE)

(21) Appl. No.: 17/776,391

(22) PCT Filed: Nov. 16, 2020
(86) PCT No.: PCT/EP2020/082212

§ 371 Date: May 12, 2022

(30) Foreign Application Priority Data
Nov. 14, 2019 (GB) 1916561.2

(51) Int. Cl. G01N 33/50; G01N 33/68

(52) U.S. Cl. CPC G01N 33/5035; G01N 33/6896; G01N 2333/4709; G01N 2333/96425

(57) ABSTRACT

Current application relates to the field of neurodegenerative diseases. More specifically, the present invention relates to screening methods to identify compounds that can reduce the production of amyloidogenic Amyloid beta fragments. Said compounds can be used in treatments of for example Alzheimer's disease.

### FIELD OF THE INVENTION

Current application relates to the field of neurodegenerative diseases. More specifically, the present invention relates to screening methods to identify compounds that can reduce the production of amyloidogenic Amyloid beta fragments. Said compounds can be used in treatments of for example Alzheimer's disease and related dementias.

### INTRODUCTION

Gamma (g)-secretases are intramembrane-cleaving proteases involved in various signalling pathways and diseases. G-secretases consist of 4 subunits. The catalytic activity of the complex is provided by presenilin (PSEN)1 or PSEN2, while three additional subunits, APH1 (A (long or short), B or C), nicastrin (NCST), and PEN-2 are needed to build a functional enzyme (De Strooper and Annaert 2010 Annu Rev Cell Dev Biol 26). G-secretase complexes are responsible for the formation of a plethora of Amyloid beta (Ab) fragments with different lengths, e.g. Ab38, Ab40, Ab42, Ab43. Several studies point to long Ab peptides (Ab42 or longer) as key players in the oligomerization and initiation of aggregation of neurotoxic or amyloidogenic Ab species, which contribute directly to the development and progression of Alzheimer's disease (AD) (Haass and Selkoe 2007 Nat Rev Mol Cell Biol 8). G-secretase inhibitors have been considered promising drug candidates against AD due to their ability to reduce Ab production. However, clinical trials have been halted due to lack of clinical efficacy and/or side effects. Indeed, recent in vitro and in vivo studies suggest that low doses of g-secretase inhibitors increase Ab production (including amyloidogenic fragments) instead of lowering it (Agholme et al 2017 Mol Cell Neurosc 85). Further basic research is thus needed to elucidate the underlying reasons and to provide new insights that will be fundamental in the development of improved therapies targeting g-secretase.

We have previously shown that g-secretase complexes comprising the catalytic subunit PSEN2 are localised in late endosomes, while those with PSEN1 are distributed more broadly in the cell (Sannerud et al 2016 Cell 166). It was surprisingly found that familial AD (FAD)-causing mutations in PSEN1 trigger a translocation of PSEN1 to late endosomes and that g-secretase complexes located at these late endosomes produce more toxic, aggregation prone Ab42 fragments. As an alternative for the failed g-secretase inhibitors, WO2018130555A1 disclosed a screening assay for g-secretase stabilizers based on the finding that instable g-secretase complexes produce more toxic Ab fragments and that complex instability is correlated with early onset AD mutations (Szaruga et al 2017 Cell 170).

### SUMMARY OF THE INVENTION

Failed clinical trials have decreased enthusiasm towards the exploration of g-secretase inhibitors in AD drug development. In current application we provide cell biological data explaining the non-efficacy of currently available g-secretase inhibitors. It is disclosed that g-secretase inhibitors besides inhibiting the catalytic activity of the g-secretase complex, induce a translocation of PSEN1/g-secretase to the late endosomes. Importantly at low concentrations of g-secretase inhibitors, the effect on PSEN1/g-secretase translocation is stronger than its catalytic inhibition, likely explaining the reported paradoxical increase in Ab production upon g-secretase inhibitor administration. These new insights are crucial for the experimental set up of future screens to identify molecules reducing the pool of amyloidogenic Ab fragments.

Therefore in a first aspect, current application provides a method to select an improved inhibitor of amyloidogenic Amyloid beta (Abeta) fragment production from a collection of gamma (g)-secretase inhibitors, comprising the steps of a) administering a g-secretase inhibitor from said collection to a cell culture expressing a g-secretase complex comprising presenilin1; b) determining the cellular localisation of said g-secretase complex in said cell culture before and after administering the g-secretase inhibitor and c) identifying said g-secretase inhibitor as an improved inhibitor of amyloidogenic Abeta fragment production if the g-secretase complex is at least 25% less translocated to the late endosomes in response to the administration of the g-secretase inhibitor of step a) compared to the translocation of the g-secretase complex observed upon administration of 100 nM DAPT or 100 nM Semagacestat.

In a second aspect, a screening method for an inhibitor of amyloidogenic Abeta fragment production is provided, comprising the steps of a) providing a cell culture expressing a g-secretase complex comprising presenilin1; b) determining the intracellular localisation of said g-secretase complex in said cell culture before and after administering a test compound to said culture; c) quantifying Abeta peptides with a length of 38, 40, 42 and/or 43 amino acids produced in said system before and after administering said test compound; d) identifying said test compound as an inhibitor of amyloidogenic Abeta fragment production if after the administration in step b) the g-secretase complex is at least 25% less translocated to the late endosomes compared to the translocation of the g-secretase complex observed upon administration of 100 nM DAPT or 100 nM Semagacestat and if the quantification in step c) reveals a statistically significant increase in the ratio of Abeta38/42, Abeta40/42, Abeta40/43 or of (Abeta38+40)/(Abeta42+43) compared to the situation before administering said test compound.

In one embodiment of both aspects, the cell culture consists of mammalian cells, particularly neuronal cells, more particularly hippocampal neurons. In another embodiment, the methods of the application are provided wherein presenilin1 is fluorescently labelled. In another embodiment, the late endosomes are visualised by a fluorescent live staining of acidified organelles, fluorescently-tagged LAMP1, antibodies to LAMP1 or lysobisphosphatidic acid. In yet another embodiment, the methods disclosed herein are provided wherein said inhibitor of amyloidogenic Abeta fragment production is a therapeutic candidate for the prevention and/or treatment of Alzheimer's disease.

### BRIEF DESCRIPTION OF THE FIGURES

FIG. 1. DAPT relocates PSEN1/g-secretase to late endosomes. (A) PSEN1 and PSEN2 double knock-out (dKO) MEFs stably expressing GFP-PSEN1 or GFP-PSEN2 were treated for 16 h with 1 μM of DAPT or DMSO and immunostained for GFP (green) and LAMP1 antibodies (red). (B) Quantification of (A) using Mander's coefficient. (C) Ratio of GFP intensity in LAMP1-positive structures over total GFP intensity in the cell, normalized to DMSO-treated cells (control=Ctrl). (D) Quantification of size area of LAMP1-positive organelles per cell showing no significant differences between DMSO and DAPT-treated cells. (E) PSEN1, PSEN2 and NCT triple KO MEFs stably rescued with NCT-SNAP and GFP-PSEN1 were treated for 16 h with 1 μM of DAPT or DMSO (Ctrl). Prior to fixation, SNAP-Cell 647-SiR was added to label NCT-SNAP (red for Merge2). After 1 h, cells were fixed and immunostained for GFP (green) and LAMP1 (red for Merge2). Two merged images are shown. Merge1 corresponds to GFP-PSEN1 (green) and NCT-SNAP (red), and Merge2 GFP-PSEN1 (green) and LAMP1 (red). (F) Rat primary hippocampal neurons expressing GFP-PSEN1 (at 7 days in vitro) were treated with 1 μM DAPT or DMSO (Ctrl), fixed and processed for immunolabeling with anti-GFP (green) and anti-PSEN2 (red). (G) PSEN1 KO MEFs rescued with GFP-PSEN1 were treated for 16 h with 1 μM of DAPT, fixed, and immunostained for GFP (green) and LBPA (red). Scale bars=10 μm. Insets show magnifications of the regions indicated by a dotted box. All graphs show the median with 95% confidence interval (n=3, ˜20 cells per experiments; p-values are calculated using Mann-Whitney test; ns means non-significant).

FIG. 2. G-secretase inhibitors re-route PSEN1/γ-secretase to late endosomes. (A) MEF PSEN1 knock-out cells stably expressing GFP-PSEN1 were treated with increasing dose of DAPT. After 16 h cells were fixed and processed for immunolabeling using anti-GFP (green) and anti-LAMP1 antibodies (red). Insets zoomed areas indicated by a dotted box. Scale bar=10 μM. (B) Quantification of (A) by measuring the ratio of the GFP intensity in LAMP1-positive structure over total GFP intensity in the cell for each condition. Data were normalised to control (DMSO). Mean+SEM, n=2 or 3, ˜20 cells per experiment). (C, D, E) Dose-response curves for the re-location of GFP-PSEN1 to late endosomes for cells treated with (C) L-685,458, (D) semagacestat (Sema) or (E) GSM-4.

FIG. 3. G-secretase inhibitors do not relocate PSEN1/g-secretase for lysosomal degradation. PSEN1 KO MEFs rescued with GFP-PSEN1 were treated for 16 h with different drugs as indicated, fixed and immunostained for GFP (green) and LAMP1 (red). Insets: zoom of boxed areas. Scale bar=20 μm. Ratio of the mean GFP intensity in LAMP1-positive structures over total mean GFP intensity of the cell is calculated. (A) Effect of DAPT (1 μM) and DAPT wash-out with complete medium alone or supplemented with BafilomycinA1 (BafA1, 100 nM) or Leupeptin (Leup, 20 μM). Values are normalized to control (DMSO treated cells). Mean+SEM, n=3, ˜20 cells per experiment. Each p-value is calculated to the DAPT-treated sample using Mann-Whitney test. (B) Cells treated with BafA1 alone (100 nM) or DAPT (1 μM) with Baf1A (100 nM), for 16 h. Values are normalized to BafA1 alone (mean+SEM, n=3, ˜20 cells per experiment). (C) Cell treated with DAPT (1 μM) and cycloheximide (CHX; 30 μg/ml) or DAPT wash-out in the presence of CHX (30 μg/ml). Values are normalized to control (DMSO) (mean+SEM, n=3, ˜20 cells per experiment).

FIG. 4. G-secretase inhibitors affect recycling of PSEN1/g-secretase from endosomes to the cell surface. PSEN1 KO MEFs, stably expressing EGFP-PSEN1 were processed for cell surface biotinylation using cleavable biotin. After biotinylation, cells were incubated at 37° C. without or with DAPT for (A, B) 5, 10, 30 and 60 min (A, B) or for 16 h (C, D). As a negative control, cells were also kept at 4° C. (NR: non-reduced; 0 time point: reduced sample). After each time point, the remaining biotin at the cell surface was cleaved off at 4° C. and next cells were lysed. Internalised biotinylated proteins were pulled down using streptavidin beads. (A, C) Representative Western blots are shown for NCT and transferrin receptor (TfR). Quantification is shown in (B, D). Graphs represent the mean+SEM of two to three experiments. Overall, DAPT does not alter internalization but results in an increased pool (16 h, D) of internalized NCT representing accumulated PSEN1/g-secretase complexes. P-values are calculated using Mann-Whitney test between control and DAPT sample.

FIG. 5. Inhibitor dose-effect on Ab production. PSEN1 KO MEFs stably rescued with GFP-PSEN1 and transfected with C99-3×FLAG (6 h) were treated with different concentrations of (A, B) DAPT, or (C) L-685-458 as indicated. After 16 h, condition media and cell lysates were analyzed by Western blot using 82E1 monoclonal antibody to detect AP. Representative blot is shown in B for DAPT treatment and quantification is shown in (B): mean+SEM, n=3 independent experiments. (C) PSEN1 KO MEFs. Quantification for L-685-458 is shown in (C), n=1.

FIG. 6. Familial Alzheimer's disease (FAD) mutations in PSEN1 differently affect g-secretase localization in late endosomes. Ratio of GFP intensity in LAMP1 endosomes over total GFP signal in PSEN1 KO MEFs stably rescued with GFP-PSEN1 wild-type (Wt) or the indicated FAD-PSEN1 mutants and treated with DMSO or DAPT (1 μM) 16 h. Histogram shows median with 95% confidence interval of a representative experiment with 20 to 40 cells. p-Values are calculated using the Mann-Whitney test and compared to PSEN1-Wt, DMSO treated.

FIG. 7 shows a Western blot illustrating the level of amyloid precursor protein (APP) full length (FL), Cadherin (Cad) FL, Cad C-terminal fragment (CTF) and APP CTF upon administration of 12 compounds. Cad CTF and APP CTF accumulate in the presence of g-secretase inhibitors C1, C2, C4, C8 and the positive control DAPT demonstrating the inhibitory effect of those compounds on the enzymatic activity of the g-secretase complex. DMSO is used as negative control.

FIG. 8 shows the effect of 12 compounds on the intracellular localisation of the PSEN1/g-secretase complex. Rel MFI (y-axis) means the ratio of the mean GFP intensity in LAMP1-positive structures (late endosomes) over total GFP intensity in the cell normalized to the negative control. Compounds were used in 1 μM concentration. G-secretase inhibitors (DAPT as positive control, C1, C2, C4 and C8) induce a translocation of the PSEN1/g-secretase complex to the late endosomes while none of the g-secretase stabilizers (C3, C5-C7, C9-12) did. DMSO is used as negative control.

FIG. 9 is a dose-response curve (0.1-1000 nM) of the translocation effect of g-secretase inhibitors C1, C2, C4 and C8. The Y-axis shows the ratio of the mean GFP intensity in LAMP1-positive structures (late endosomes) over total GFP intensity in the cell normalized to the negative control.

FIG. 10 is a dose-response curve (0.1-1000 nM) on the Cadherin CTF accumulation of the g-secretase inhibitors C1, C2, C4 and C8. The concentration in the X-axis is mentioned in nM. The Y-axis is the fold increase of the Cadherin CTF accumulation compared to the 0 nM level.

FIG. 11 is a dose-response curve (0.1-1000 nM) on the APP CTF accumulation of the g-secretase inhibitors C1, C2, C4 and C8. The concentration in the X-axis is mentioned in nM. The Y-axis is the fold increase of the APP CTF accumulation compared to the 0 nM level.

### DETAILED DESCRIPTION

As used herein, each of the following terms has the meaning associated with it in this section. The articles “a” and “an” are used herein to refer to one or to more than one (i.e. to at least one) of the grammatical object of the article. By way of example, “an element” means one element or more than one element. “About” as used herein when referring to a measurable value such as an amount, a temporal duration, and the like, is meant to encompass variations of ±20% or ±10%, more particularly ±5%, even more particularly ±1%, and still more particularly ±0.1% from the specified value, as such variations are appropriate to perform the disclosed methods. The term “abnormal” when used in the context of organisms, tissues, cells or components thereof, refers to those organisms, tissues, cells or components thereof that differ in at least one observable or detectable characteristic (e.g. age, treatment, time of day, etc.) from those organisms, tissues, cells or components thereof that display the “normal” (expected) respective characteristic. Characteristics which are normal or expected for one cell or tissue type, might be abnormal for a different cell or tissue type. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. Unless specifically defined herein, all terms used herein have the same meaning as they would to one skilled in the art of the present invention. Practitioners are particularly directed to Sambrook et al. 2012 Molecular Cloning: A Laboratory Manual, 4th ed., Cold Spring Harbor Press, Plainsview, N.Y.; and Ausubel et al. 2012 Current Protocols in Molecular Biology (Supplement 100), John Wiley & Sons, New York, for definitions and terms of the art. The definitions provided herein should not be construed to have a scope less than understood by a person of ordinary skill in the art.

Plaques, composed of the aggregation-prone Amyloid beta (Ab) peptides, are key pathological hallmarks of Alzheimer's disease (AD). Ever since the amyloid cascade hypothesis was proposed in 1991 (Hardy and Allsop 1991 Trends Pharmacol Sci 12), much research on AD pathogenesis has focused on the mechanisms behind Ab generation and degradation. In line with this, several drug candidates have been developed, aiming at reducing Ab production especially amyloidogenic Ab fragments by inhibiting or modulating the key enzymes involved in the production of Ab. Ab is generated by sequential cleavage of the amyloid precursor protein (APP) by beta-secretase followed by gamma-secretase (Blennow et al 2006 Lancet 368), and therefore both beta- and g-secretase inhibitors have been proposed as possible treatments for AD. Unfortunately, g-secretase inhibitors failed to reach clinical testing or failed in clinical trials, one reason being non-efficacy. Indeed, as the drug concentration in plasma changes during the treatment, also Ab levels in blood fluctuate. Both in vivo and in vitro studies have shown that low doses of g-secretase inhibitors unlogically increase Ab levels, instead of decreasing them (Barnwell et al 2014 PLoS One e91531; Agholme et al 2017 Mol Cell Neurosc 85).

In the current application at least one reason for the non-efficacy of the failed g-secretase inhibitors is disclosed. The inventors of current application found that those inhibitors induce a translocation of the PSEN1/g-secretase complex to the late endosomes, a subcellular compartment known to promote the formation of amyloidogenic Ab fragments (Sannerud et al 2016 Cell 166). Hence, a drug discovery trajectory aiming at developing compounds reducing the production of amyloidogenic Ab fragments should comprise a further selection step to ensure that potentially interesting compounds do not induce the unwanted translocation of the g-secretase complex. Therefore, in a first aspect, a method to select inhibitors more particularly improved inhibitors of amyloidogenic Ab fragment production is provided. Said method starts from a collection of compounds that in previous assays demonstrated the ability to inhibit the g-secretase complex with the aim to produce less amyloidogenic Ab fragments in patients. Said collection can thus be seen as a collection of g-secretase inhibitors. Said collection is tested in an in vitro system, preferably a cell culture expressing a g-secretase complex comprising PSEN1. In a most ideal and most likely in a therapeutic unrealistic situation, the method of the first aspect would select or identify a g-secretase inhibitor from said collection as an improved inhibitor of amyloidogenic Ab fragment production if the g-secretase complex is not translocated to the late endosomes at all after administering said g-secretase inhibitor. However, any reduced translocation towards the late endosomes will be beneficial. Therefore, in particular embodiments, a g-secretase inhibitor from said collection will be identified or selected as an improved inhibitor of amyloidogenic Ab fragment production if the g-secretase complex is statistically significantly less translocated to the late endosomes compared to a control situation. In other particular embodiments, a g-secretase inhibitor from said collection will be selected as an improved inhibitor of amyloidogenic Ab fragment production if the g-secretase complex is at least 10%, 25%, 40%, 50%, 60%, 70%, 80%, 90% or 95% less translocated to the late endosomes compared to a control situation. A suitable control situation is the translocation induced by known g-secretase inhibitors such as DAPT or Semagacestat. Therefore, in further embodiments, said control situation is the translocation of the g-secretase complex observed upon administration of between 10 and 1000 nM DAPT or of between 100 and 1000 nM Semagacestat, more particularly upon administration of 100 nM DAPT or 100 nM Semagacestat. Other suitable control situations especially adapted to library screens in multi-well plates (see further) is the mean translocation of all compounds tested in 1 or more multi-well plates. Therefore, in other embodiments, said control situation is the mean translocation of the g-secretase complex induced by all compounds tested in 1 or more multi-well plates. In further embodiments, the translocation is determined by the change in late endosomal localisation value, wherein said value is measured by the ratio of the presenilin1 signal in the late endosomes to the total cellular presenilin1 signal. Screening for improved g-secretase complex inhibitors as described herein thus means that said late endosomal localisation value should be as low as possible. For practical reasons the presenilin1 signal in the late endosomes in a mock situation is set at 1. In this case the late endosomal localisation value upon administering an improved g-secretase inhibitor should remain below 2, more particularly below 1.5, below 1.4 or below 1.3, most particularly below 1.25, below 1.2, below 1.15, below 1.1 or even most particularly remain at 1.

In other particular embodiments, a g-secretase inhibitor from said collection will be selected as an improved inhibitor of amyloidogenic Ab fragment production if the translocation of the g-secretase complex to the late endosomes is at most 50%, 25%, 20%, 15%, 10%, 5% or 2% more compared to the situation before the g-secretase inhibitor was administered.

The methods provided in the first aspect are equivalent to screening methods for g-secretase inhibitors that prevent the late endosomal localisation of g-secretase complexes in a cell or prevent the translocation of g-secretase complexes to the late endosomes in a cell. Those g-secretase inhibitors can be seen as improved or optimized g-secretase inhibitors as it is herein disclosed that the translocation to the late endosomes should be prevented. Therefore, said improved g-secretase inhibitors are equivalent to non-translocating g-secretase inhibitors or more particularly g-secretase inhibitors that do not translocate g-secretase complexes or do not translocate g-secretase complexes to the late endosomes or prevent the late endosomal localisation of g-secretase complexes. In one embodiment, said g-secretase complexes are PSEN1/g-secretase complexes. In another embodiment, said improved g-secretase inhibitors are Ab lowering or reducing compounds.

The terms “gamma-secretase”, “g-secretase”, “gamma-secretase protein complex” and “gamma-secretase complex” refer to a protein complex used in the present application comprising at least four protein molecules, where at least one of the protein molecules provides a catalytic site for cleavage of a polypeptide substrate having a g-secretase cleavage sequence, and wherein the protein molecules are PSEN1 or PSEN2, Aph1a or Aph1b or Aph1c, NCT, and/or PEN2. The amyloid precursor protein (APP) is an example of such substrate. In a particular embodiment of current application, g-secretase is PSEN1/g-secretase referring to a g-secretase complex comprising PSEN1.

A “g-secretase inhibitor” as used herein refers a chemical or biological molecule that inhibits the catalytic activity of the g-secretase complex. In a particular embodiment, said g-secretase inhibitor inhibits the catalytic activity of PSEN1/g-secretase complexes. Presenilin-1 is a presenilin protein that is encoded by the PSEN1 gene (OMIM: 104311). The following compounds are non-limiting examples of g-secretase inhibitors: DAPT (GSI-IX), RO4929097, Semagacestat (LY450139), Avagacestat (BMS-708163), Dibenzazepine (YO-01027), LY-411-575, IMR-1, L-684-458, FLI-06, Crenigacestat (LY3039478), Nirogacestat (PF-03084014, PF-3084014), MK-0752, NGP 555, CHF 5074.

The “late endosomes” or the “late endosomal compartment” refers to an intracellular or subcellular compartment that functions as an important sorting station in the endocytic pathway. They are prelysosomal endocytic organelles defined by the time it takes for endocytosed macromolecules to be delivered to them. Late endosomes are differentiated from early endosomes by their lower lumenal pH, different protein composition and association with different small GTPases of the Rab family. They are differentiated from lysosomes, because late endosomes are enriched in the two mannose 6-phosphate receptors (MPRs), whereas lysosomes have neither. The late endosomal compartment can be visually identified using suitable cell biological markers comprising but not limited to for example Rab7, LAMP1 and lysobisphosphatidic acid (LBPA).

The methods of the first aspect comprise the steps of a) administering a g-secretase inhibitor to a cell culture comprising or expressing a g-secretase complex comprising presenilin1 (PSEN1), b) determining or monitoring the cellular localisation of said g-secretase complex in said culture and c) identifying said g-secretase inhibitor as an improved or optimized g-secretase inhibitor if after the administration step the g-secretase complex is not translocated to the late endosomes or the sorting of the g-secretase complex to the late endosomes is inhibited. Alternatively, said methods are provided wherein step c) identifies said g-secretase inhibitor as an improved or optimized g-secretase inhibitor if under the same test conditions in the same system the g-secretase complex is less deviated to the late endosomes compared to DAPT or Semagacestat treatment. In a particular embodiment, less deviated compared to DAPT or Semagacestat means statistically significantly less. In another particular embodiment, less deviated compared to DAPT or Semagacestat means at least 10%, 25%, 40%, 50%, 60%, 70%, 80%, 90% or 95% less. In another embodiment, said comparison is done with at least 10 nM DAPT or at least 100 nM Semagacestat, more particularly at a concentration of DAPT between 10 and 1000 nM or of between 100 and 1000 nM of Semagacestat, more particularly upon administration of 100 nM DAPT or 100 nM Semagacestat.

In the methods of the first aspect, a g-secretase inhibitor or a collection of compounds of which the g-secretase inhibition was previously demonstrated, is used to start the screening method with. Said methods can thus be seen as secondary or tertiary screens in a drug discovery project to further select the most promising g-secretase inhibitor(s). However, current disclosure is not limited thereto. The insights can also be used for a primary screening method starting with a library of unknown compounds. Therefore, in a second aspect, a screening method is provided for compounds that inhibit or reduce amyloidogenic Abeta fragment production or for inhibitors of amyloidogenic Abeta fragment production. Said method comprises the steps of:
a) providing a cell culture comprising a g-secretase complex, more particularly a fluorescently-tagged g-secretase complex;
b) determining the intracellular localisation of said g-secretase complex in said system after administering a test compound to said culture and optionally before said administration;
c) quantifying Abeta peptides with a length of 38, 40, 42 and/or 43 amino acids produced in said system before and after administering said compound;
d) identifying said test compound as an inhibitor of amyloidogenic Abeta fragment production if after the administration in step b) the g-secretase complex is at least 10%, 25%, 40%, 50%, 60%, 70%, 80%, 90% or 95% less or statistically significantly less translocated to the late endosomes compared to a control situation and if the quantification in step c) reveals a statistically significant increase in the ratio of Ab38/Ab42, Ab40/Ab42, Ab40/Ab43 or of Ab(38+40)/Ab(42+43) compared to the situation before administration of said compound.

In a particular embodiment, said g-secretase complex comprises presenilin1. In other particular embodiments, said control situation of step d) is the translocation of the g-secretase complex to the late endosomes observed upon administration of between 10 and 1000 nM DAPT or of between 100 and 1000 nM Semagacestat, more particularly upon administration of between 20 and 100 nM DAPT or between 20 and 100 nM Semagacestat. In yet other embodiments, said control situation is the mean translocation of the g-secretase complex to the late endosomes induced by all compounds tested in 1 or more experiments.

In other particular embodiments, a test compound can also be selected as an inhibitor of amyloidogenic Ab fragment production if in step d) the translocation of the g-secretase complex to the late endosomes is at most 50%, 25%, 20%, 15%, 10%, 5% or 2% more compared to the situation before the test compound was administered.

In other particular embodiments of the methods of the second aspect, said statistically significant increase in the ratio of Ab38/Ab42, Ab40/Ab42, Ab40/Ab43 or of Ab(38+40)/Ab(42+43) is an at least 10%, 25%, 40%, 50%, 60%, 70%, 80%, 90%, a 2-fold, 5-fold or 10-fold increase.

“Statistically significant” as used in current application refers to the likelihood that a result is not attributed to chance. Statistical hypothesis testing is thus a way of quantifying the unlikely-ness of an experimental result. As statistical analysis is part of every (cell) biological experiment, the skilled person is also skilled in statistics and knows that a result is statistically significant if the Null hypothesis (which means there is no difference) is rejected. A result is statistically significant, hence the null hypothesis is rejected, if the probability of obtaining a result by chance is less than a pre-defined significance level (p-value). Consequently, when the test result exceeds the p-value, the null hypothesis is accepted, and the result is not statistically significant. The significance level p is typically set at 5%. In one embodiment, said p-value is 1%. In a most particular embodiment, p is 0.1%.

In one embodiment, the ratio of Ab38/Ab42 peptides will be determined, while in an alternative embodiment a ratio of Ab40/Ab43 will be determined, and in another embodiment the ratio of Ab40/Ab42 will be defined, and finally, also the sum of the “shorter” and “longer” peptides can be determined by Ab(38+40)/Ab(42+43). When an increased ratio is obtained, the resulting amount of shorter Ab peptides will be higher than the resulting amount of “less-processed” or longer Ab peptides, which indicates that the test compound reduces the production of amyloidogenic Ab fragments.

“Amyloidogenic” as used herein refers to producing or tending to produce amyloid oligomers and deposits. “Amyloidogenic Ab fragments” are those Ab fragments that lead to amyloid plaque formation or alternatively phrased “oligomeric Ab fragments”. Ab oligomers are those initial Ab fragments that lead to neurotoxic effect, in particular synaptic toxicity. In a particular embodiment, said amyloidogenic Ab fragments are Ab42, Ab43, Ab45 and/or Ab46, even more particularly Ab42 and/or Ab43. Detection and quantification of said produced Ab peptides in said system is in one embodiment obtained via “immune-based assays” or “immune-based detection” or “immune-based quantification”, used interchangeably herein, which refer to the most broadly used bio-detection technologies that are based on the use of antibodies, and are well known in the art. Antibodies are highly suited for detecting small quantities of specific peptides or proteins in the presence of a mixture of peptides or proteins. Said “immune-based detection” refers to a biochemical binding assay involving binding between antibodies and antigen, which measures the presence or concentration of a substance in a sample, such as a biological sample, or an in vitro sample, using the reaction of an antibody to its cognate antigen, for example the specific binding of an antibody to a specific Ab peptide. Both the presence of the antigen or the amount of the antigen present can be measured. Examples of immunoassays are enzyme linked immunosorbent assays (ELISAs), enzyme linked immunospot assay (ELISPOT), immunobead capture assays, Western blotting, gel-shift assays, protein arrays, multiplexed bead arrays, magnetic capture, fluorescence resonance energy transfer (FRET), a sandwich assay, a competitive assay, an immunoassay using a biosensor, an immunoprecipitation assay etc. Antibodies are currently available to detect and distinguish each type of resulting Ab peptide relevant for determination of said ratio: Ab38, Ab40, Ab42, Ab43, . . . can be specifically detected and quantified, for instance via ELISA applying specific antibodies as for example explained in WO2018130555A1.

Detection and quantification of said produced Ab peptides in said system can also obtained in another embodiment via “mass-spectrometry” or “MS-based detection” or “mass-spectrometry-based quantification”, used interchangeably herein, which refer to detection/quantification methods specifically defining the desired Ab peptides, such as Ab38, Ab40, Ab42, Ab43 and explained in for example WO2018130555A1.

The methods of the second aspect and of its embodiments are thus a further elaboration of the methods of the first aspect. In the former, not a preselected gamma-secretase inhibitor is used but an unknown compound and as it still has to be evaluated whether said compound would be functional as a gamma-secretase inhibitor, the methods of the second aspect further comprise a step of quantifying the Ab peptides with a length of 38, 40, 42 and/or 43 amino acids produced in the cell culture before and after administering said test compound. Consequently, the final selection step adds a second criterium besides the reduced translocation of the g-secretase complex to late endosomes, i.e. the quantification step should reveal an increase in the ratio of Ab38/Ab42, Ab40/Ab42, Ab40/Ab43 or Ab(38+40)/Ab(42+43) upon administration of said test compound.

The term “compound” is used herein in the context of a “test compound” or a “drug candidate compound” described in connection with the methods of the present invention. As such, these compounds comprise organic or inorganic compounds, derived synthetically or from natural resources. The compounds include polynucleotides, lipids or hormone analogues that are characterized by low molecular weights. Other biopolymeric organic test compounds include small peptides or peptide-like molecules (peptidomimetics) comprising from about 2 to about 40 amino acids and larger polypeptides comprising from about 40 to about 500 amino acids, such as antibodies, antibody fragments or antibody conjugates.

In a further embodiment of all embodiments of the methods of the first and second aspect, the cell culture that is used in the methods is an in vitro cell system. In another further embodiment, said cell culture or said in vitro cell system comprises or consists of mammalian cells, more particularly human cells. In another further embodiment, said mammalian or human cells are neuronal cells derived from an extended pluripotent stem (EPS) cell line or an induced pluripotent stem (iPS) cell line via differentiation. The skilled person is familiar with the production of EPS and/or iPS cell lines and the differentiation into neuronal cells.

In other further particular embodiments, the cells described above expressing a g-secretase complex comprising presenilin1 (PSEN1) comprise a PSEN1 that is tagged or fused with a detectable marker. Examples of detectable markers for tagging PSEN1 are described herein further. However, the methods of current application are not limited to labelled PSEN1 as the cellular localisation of PSEN1 can also be determined using directly or indirectly labelled anti-PSEN1 antibodies. How the cellular localisation of PSEN1/g-secretase can be determined in the cells of current application is known to the person skilled in the art. Additionally the experimental part of current application explains this explicitly.

In particular embodiments of the methods of the second aspect the present invention provides a system and method to screen and identify compounds that reduce the formation of amyloidogenic Ab fragments and at the same time selectively inhibit PSEN1/g-secretase complexes to sort to late endosomes. In one embodiment, the systems and methods comprise high content screening (HCS) of suitable compounds. In some instances, HCS is a screening method that uses live cells to perform a series of experiments as the basis for high throughput compound discovery. Typically, HCS is an automated system to enhance the throughput of the screening process. However, the present invention is not limited to the speed or automation of the screening process.

In another embodiment of the invention, the HCS assay provides for a high throughput assay. Preferably, the assay provides automated screening of thousands of test compounds. Compounds tested in the screening methods of the present invention are not limited to the specific type of the compound. In one embodiment, entire compound libraries are screened. Compound libraries are a large collection of stored compounds utilized for high throughput screening. Compounds in a compound library can have no relation to one another. As would be understood by one skilled in the art, the methods of the second aspect are not limited to the types of compound libraries screened. Non-limiting examples of compound libraries include the sets from LOPAC, Chembridge, Maybridge, LifeChemicals and the NIH Clinical Collection.

Alternatively, compounds in a compound library can have a common characteristic. For example, a hypothetical compound library may contain all compounds known to inhibit the catalytic activity of g-secretase complexes or to reduce the formation of amyloidogenic Ab fragments in a cell or in in vitro conditions. The methods of the first aspect for example use such a library or a pool or collection of compounds of which it is known that they inhibit g-secretase activity, more particularly PSEN1/g-secretase activity.

In both aspects of the invention, the test compound may be added to the assay to be tested by any suitable means. For example, the test compound may be injected into the cells of the assay, or it can be added to the nutrient medium and allowed to diffuse into the cells. In situations where “high-throughput” modalities are preferred, it is typical that new chemical entities with useful properties are generated by identifying a chemical compound (called a “hit compound”) with some desirable property or activity, and evaluating the property of those compounds. A non-limiting example of a high-throughput screening assay is to array the invention to 96, 384, 1536, etc. well or slot format to enable a full high throughput screen.

In one embodiment, high throughput screening methods involve providing a library containing a large number of compounds (candidate compounds) potentially having the desired activity. Such “combinatorial chemical libraries” are then screened in one or more assays, as described herein, to identify those library members (particular chemical species or subclasses) that display a desired characteristic activity. The compounds thus identified can serve as conventional “hit compounds” or can themselves be used as potential or actual therapeutics.

The system and methods of the invention are based upon the detection of the localization of PSEN1/g-secretase complexes in a living cell or fixed cell. In one embodiment, the methods of the application comprise the acquisition of images of cells to detect PSEN1/g-secretase complexes localization. Localization of PSEN1/g-secretase complexes is made through the detection of a signal corresponding to PSEN1/g-secretase complexes. In one embodiment, the screening methods of the invention comprise the use of cells that do not natively express PSEN1/g-secretase complexes. In another embodiment the screening methods of the invention comprise cell lines which have been mutated in PSEN1 and do not express a normal functional or wild type PSEN1. In one embodiment, cells are genetically modified to express PSEN1/g-secretase complexes. The present invention is not limited to cells expressing full-length PSEN1/g-secretase complexes. In one embodiment, cells of the methods express PSEN1/g-secretase complexes wherein the PSEN1 is tagged with a detectable marker, for example fluorescently tagged PSEN1. Non-limiting examples of fluorescent tags include green fluorescent protein (GFP), cyan fluorescent protein (CFP), yellow fluorescent protein (YFP), red fluorescent protein (RFP), orange fluorescent protein (OFP), eGFP, mCherry, hrGFP, hrGFPII, Alexa 488, Alexa 594, and the like. Fluorescent tags may also be photoconvertible such as for example kindling red fluorescent protein (KFP-red), PS-CFP2, Dendra2, CoralHue Kaede and CoralHue Kikume or photoactivable such as photoactivatable GFP and photoactivatable Cherry and the like. However, the invention should not be limited to a particular label. Rather, any detectable label can be used to tag PSEN1.

In a further embodiment of the methods of the first and second aspect, the screen uses a cell or cell population modified to express PSEN1/g-secretase complexes and/or other proteins of interest. In one embodiment, the cell or cell population is modified by administering an expression vector encoding the protein of interest. As would be understood by those skilled in the art, the expression vector used to modify the cell or cell population of the screen includes any vector known in the art such as cosmids, plasmids, phagemid, lentiviral vectors, adenoviral vectors, retroviral vectors, adeno-associated vectors, and the like. The expression vector may be provided to a cell in the form of a viral vector. Viral vector technology is well known in the art and is described in virology and molecular biology manuals. Viruses, which are useful as vectors include, but are not limited to, retroviruses, adenoviruses, adeno-associated viruses, herpes viruses, and lentiviruses. A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used. In one embodiment, the cell or cell population of the screen is administered a lentiviral vector encoding PSEN1. Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

In one embodiment, the cells of the screen are modified to transiently express PSEN1/g-secretase complexes. In another embodiment, the cells of the screen are modified for the stable expression of PSEN1/g-secretase complexes. For example, in one embodiment, a cell line which stably expresses PSEN1, is generated and maintained under standard culturing protocols known in the art. In one embodiment, a cell of the screen comprises nucleic acids encoding PSEN1.

In one embodiment, the present invention is related to screening methods comprising the automated detection of the cellular localization of proteins. In one embodiment, the localization of PSEN1/g-secretase complexes, or other elements of interest, is determined from images taken of cells expressing PSEN1, or other elements of interest. The localization of PSEN1, or other elements of interest, may be determined in the live cell of the assay, or alternatively after the cell has been fixed. The present invention is not limited to the type or mode of microscopy utilized in imaging of the cells of the screen. In one embodiment, acquired images obtained through standard fluorescent microscopy techniques known in the art, detects the localization of the fluorescent signal in a cell, thereby detecting the localization of PSEN1 within a cell. The present invention shows that upon administration of g-secretase inhibitor, PSEN1 shuttles into late endosomes. Thus, in these conditions, images of the cell would demonstrate PSEN1 localization in late endosomes as shown in the Example section. Therefore, in embodiments wherein cells express fluorescently tagged PSEN1, images of the cells exhibit a darker cytoplasm and a brighter late endosomal compartment. As would be understood by those skilled in the art, full length PSEN1 protein, or portions thereof, can be used in the screening methods of the invention. For example, in one embodiment, cells of the screen comprise full length PSEN1. In another embodiment, cells of the invention comprise only specific regions of PSEN1, for example the N-terminal fragment or C-terminal fragment or N-terminal/C-terminal functional dimers. As explained previously, current application is not limited to cells comprising tagged or fluorescently tagged PSEN1, as PSEN1 localisation can be determined as reliably with anti-PSEN1 antibodies.

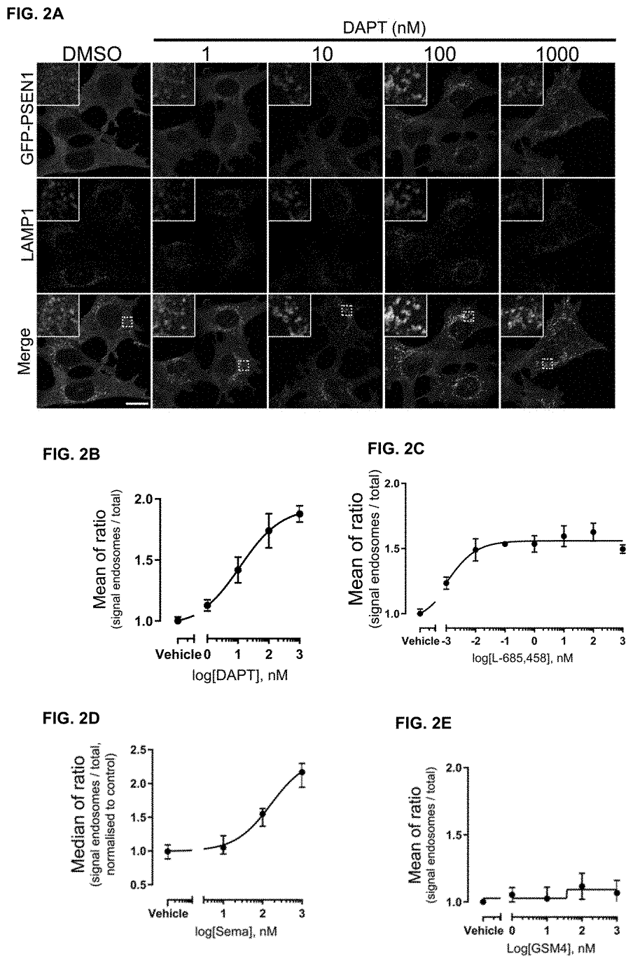

In one embodiment of the invention, localization of PSEN1 is quantitatively determined by the calculation (which can be automated) of the proportion of PSEN1 in late endosomes to cytoplasmic and other membrane compartments, referred to as the PSEN1 signal endosomes/total ratio (e.g. as demonstrated in FIG. 2B-D). Said PSEN1 signal endosomes/total ratio is herein referred to as a “late endosomal localisation value”. In one embodiment, PSEN1 in each compartment is determined by quantifying the fluorescence due to fluorescently tagged PSEN1 in each compartment including the late endosomes. Alternatively, the late endosomal localisation of PSEN1 can be measured by the fluorescence overlap of PSEN1 with a late endosomal marker, such as for example, but not limiting to, a viable stain for acidified organelles, LAMP1 or lysobisphosphatidic acid. Untreated cells (or vehicle treated) have a relatively low PSEN1 signal endosomes/total ratio or low late endosome localisation value. In some cases, it can be beneficial towards interpretation to normalise said low ratio to 1 and measure relative late endosomal localisation values. Administration of g-secretase inhibitors such as DAPT or Semagacestat induces the translocation of PSEN1/g-secretase to the endosomes (as demonstrated in the Example section) and thus leads to an increase in said PSEN1 signal endosomes/total ratio or late endosomal localisation value compared to the absence of DAPT or Semagacestat or a mock compound.

Improved or optimized g-secretase inhibitors identified by the methods of current application will preferably not affect the late endosomal localisation value or will at least lead to a smaller increase in said late endosomal localisation value compared to the administration of DAPT or Semagacestat. Along the same line, test compounds inhibiting the formation of amyloidogenic Ab fragment and not inducing PSEN1 translocation and which will be designated as hits in the methods of the second aspect, will have a significantly lower late endosomal localisation value compared to g-secretase inhibitors such as DAPT and Semagacestat or preferably have no effect on said value at all. In certain embodiments, hits are defined as those test compounds or as those compounds that inhibit amyloidogenic Ab fragment production, that do not affect the late endosomal localisation value after administration of said test compound compared to said late endosomal localisation value before administration, or have an at least 20%, 30%, 40%, 50%, 60%, 70%, 80%, 90% or even higher reduction of the late endosomal localisation value compared to the administration to the same cells under the same conditions of between 10 and 1000 nM DAPT or between 100 and 1000 nM Semagacestat. Alternatively, hits will be defined as those compounds that increase the late endosomal localisation value measured before administration with at most 50%, 25%, 20%, 15%, 10%, 5% or 2%, or hits will be defined as those compounds that have a late endosomal localisation value which is at most 50%, 25%, 20%, 15%, 10%, 5% or 2% higher than the late endosomal localisation value before administration of said compounds.

Hence, in an embodiment of the first aspect, a method to select an improved inhibitor of amyloidogenic Amyloid beta (Ab) fragment production from a collection of g-secretase inhibitors is provided, comprising the steps of:
a) administering a g-secretase inhibitor from said collection to a cell culture expressing a g-secretase complex comprising presenilin1;
b) determining a late endosomal localisation value before and after administering the g-secretase inhibitor, wherein said localisation value is the ratio of the presenilin1 signal or level in the late endosomes and the total cellular presenilin1 signal or level in said cell culture;
c) identifying said g-secretase inhibitor as an improved inhibitor of amyloidogenic Ab fragment production if the late endosomal localisation value after administering said g-secretase inhibitor is at most 50%, 25%, 20%, 15%, 10%, 5% or 2% more than the late endosomal localisation value before administering said g-secretase inhibitor or if there is no statistically significant difference in the late endosomal localisation value before and after administering said g-secretase inhibitor. Or alternatively, in an embodiment of the first aspect, a method to select an improved inhibitor of amyloidogenic Ab fragment production from a collection of g-secretase inhibitors is provided, comprising the steps of:
a) administering a g-secretase inhibitor from said collection to a cell culture expressing a g-secretase complex comprising presenilin1;
b) determining a late endosomal localisation value before and after administering the g-secretase inhibitor, wherein the late endosomal localisation value is the ratio of the presenilin1 signal or level in the late endosomes and the total cellular presenilin1 signal or level in said cell culture;
c) identifying said g-secretase inhibitor as an improved inhibitor of amyloidogenic Ab fragment production if the change in late endosomal localisation value before and after administering said g-secretase inhibitor to said cell culture is statistically significantly lower or at least 10%, 25%, 40%, 50%, 60%, 70%, 80%, 90% or 95% lower than the change in late endosomal localisation value before and after administering a control g-secretase inhibitor selected from the list consisting of DAPT, Semagacestat and L-685-458 to said cell culture.

In a further embodiment, said control g-secretase inhibitor is DAPT or Semagacestat and wherein the late endosomal localisation values are compared between the improved and control g-secretase inhibitor over a dose range between 10 and 1000 nM, more particular at a concentration of between 20 and 100 nM.

In a particular embodiment of all method of the first and second aspect, the test compound or the g-secretase inhibitor from a collection of g-secretase inhibitors is added to the cell culture in a concentration of 1000 nM, 100 nM, 10 nM, 1 nM or 100 pM or in a concentration between 10 and 200 nM or between 50 and 500 nM or between 100 and 1000 nM or in a dose range between 100 pM and 1000 nM.

HCS assays typically comprise automated screening techniques to generate a high level of information from an experiment. In one embodiment, the system of the invention comprises numerous test compounds screened on cells cultured on a multi-well plate. Non-limiting examples of multi-well plates include a 6-well plate, a 24-well plate, a 96-well plate, a 384-well plate, a 1536-well plate, . . . . As such, each well comprises its own individual experiment detecting the response to a single test compound. Statistical analysis performed on the control wells enables the determination of the overall quality of experimentation done on the entire plate. In plates with controls determined to pass a statistical standard, test compounds that do not influence or do not increase late endosomal localisation value of PSEN1 by a pre-defined amount relative to the mean of all compounds tested on the plate, that are not acutely cytotoxic and/or fluorescent outliers are flagged as “hits” and thus as optimized or improved g-secretase inhibitors.

Examples of assay methods for identifying compounds in the context of the present invention are described in the Example section, without the purpose of being limitative. It should be clear to the skilled person that the present screening methods might be based on a combination or a series of measurements, particularly when establishing the link with Ab peptide generation. Also, it should be clear that there is no specific order in performing these measurements while practicing the present invention.

For high-throughput purposes, compound libraries may be used. Examples include, but are not limited to, natural compound libraries, allosteric compound libraries, peptide libraries, antibody fragment libraries, synthetic compound libraries, etc.

Assays can be performed in eukaryotic cells, advantageously in mammalian cells, such as human cells, such as neuronal cells. Appropriate assays can also be performed in prokaryotic cells, reconstituted membranes, and using purified proteins in vitro.

In a third aspect of the application, a screening method is provided to identify an inhibitor of amyloidogenic Ab fragment production, comprising the steps of:
a) providing a cell culture expressing a g-secretase complex, wherein said g-secretase complex is localised in late endosomes of said cells;
b) administering a test compound to said cell culture;
c) monitoring the cellular localisation of the g-secretase complex in said culture, wherein under the same test conditions in the same cell culture without the test compound, a deviation in the late endosomal localisation of the g-secretase complex identifies said test compound as an inhibitor of amyloidogenic Ab fragment production.

Said deviation in step c) refers to a shift in subcellular localisation of the g-secretase complex more particularly the PSEN1/g-secretase complex, resulting in a statistically significant less late endosomal localisation. Determining said shift can be done using the late endosomal localisation value as described above. In one embodiment, said screening method is provided comprising the steps of a) administering a test compound to a cell culture expressing a g-secretase complex comprising presenilin1 wherein said g-secretase complex is localised in late endosomes of said cells, b) determining the cellular localisation of presenilin1 in said culture with and without said test compound and c) identifying said test compound as an inhibitor of amyloidogenic Ab fragment production if the presenilin1 localisation at late endosomes is statistically significantly less or at least 10%, 20%, 30%, 40%, 50%, 60%, 70%, 80%, 90% or 95% less in the presence of the test compound compared to the situation in the absence of the test compound. In a particular embodiment, late endosomal localisation of PSEN1/g-secretase in the cell culture of step a) is obtained by a mutation in PSEN1. In a particular embodiment, said mutation is selected from the list consisting of L166P, G384A and P88L. Said mutations are those as described in Szaruga et al (2017 Cell 170). All embodiments, alternatives or embodiments of the first and second aspect as described above also apply to the methods of the third aspect.

In a fourth aspect of the application, a mammalian cell devoid of endogenous Nicastrin (NCST), PSEN1 and PSEN2 protein production is provided, said cell comprising a labelled NCST and PSEN1 and/or PSEN2 protein. Nicastrin (NCST) (OMIM: 605254) is a protein that is part of the gamma-secretase protein complex and that interacts with PSEN1 (OMIM: 104311) and PSEN2 (OMIM: 600759).

“Devoid” means “lacking” or “defective in”. “Endogenous” as used herein refers to the original or native or non-modified. Hence, said animal cell devoid of endogenous Nicastrin (NCST), PSEN1 and PSEN2 protein production is a triple knock-out of NCST, PSEN1 and PSEN2. Knocking-out gene function or preventing protein production can be obtained in several ways. The skilled person is well aware of these ways which include but are not limited to genetic insertion or deletion mutations which can be obtained by mutagenesis or for example by endonuclease technology as CRISPR/Cas9. The labelled NCST, PSEN1 and PSEN2 are expressed at such level that they can complement the lack of endogenous expression of those proteins. Labelling allows the visualisation of the different subunits of the gamma-secretase complex in a cell. Preferably, the two or three different subunits are differently labelled. The protein can be tagged or labelled with a detectable marker. Examples of detectable markers for protein tagging are described herein above. In one embodiment, said mammalian cells are neuronal cells, even more particularly hippocampal neurons. In yet another embodiment, said neuronal cells are derived from an extended pluripotent stem (EPS) cell line or an induced pluripotent stem (iPS) cell line via differentiation. The skilled person is familiar with the production of EPS and/or iPS cell lines and the differentiation into neuronal cells. In a most particular embodiment, said mammalian cells are human cells.

The following examples are intended to promote a further understanding of the present invention. While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

### EXAMPLES

### Example 1. Gamma-Secretase Inhibitors Redirect PSEN1/g-Secretase to Late Endosomes

We demonstrated previously that PSEN2/g-secretase is targeted to LAMP1-positive endosomes (Sannerud et al 2016 Cell 166). This restricted localization of PSEN2/g-secretase is due to a conserved sorting motif in its N-terminus that is absent in PSEN1. PSEN1/g-secretase has no marked organelle enrichment and localizes in multiple post-Golgi compartments including the cell surface, sorting and recycling endosomes. However, surprisingly, we found that some familial AD mutations, including L166P and G384, re-located PSEN1/g-secretase to LAMP1-positive compartments, highly reminiscent of PSEN2/g-secretase localization. This correlated with an almost complete shift from Ab40 to Ab42 production, resulting in the highest increase in Ab42/40 ratio compared to other FAD-PSEN1 mutations (Sannerud et al 2016 Cell 166).

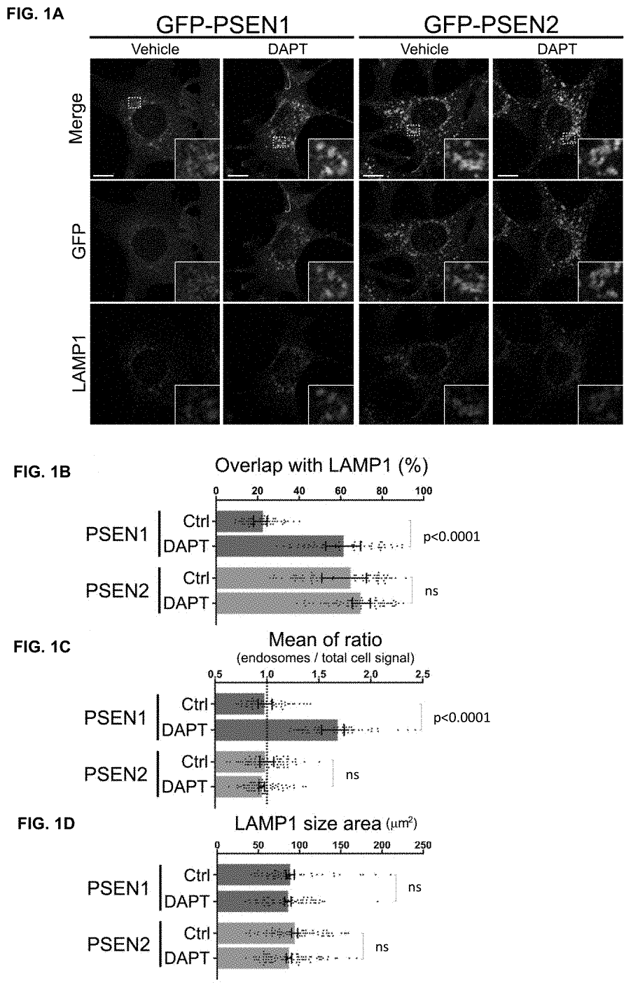

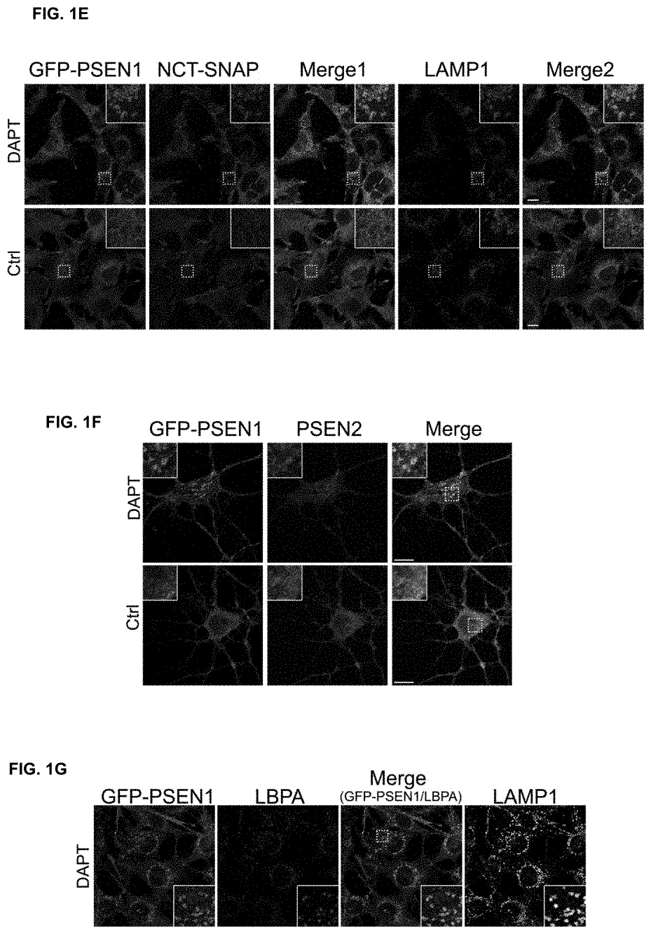

We now questioned whether g-secretase inhibitors would affect subcellular distribution of g-secretase complexes as well. We made use of mouse embryonic fibroblasts (MEFs) in which both PSEN1 an PSEN2 are knocked-out (dKO), rescued with either EGFP-PSEN1 or EGFP-PSEN2 (FIG. 1A). Western blot analysis of cell lysates confirmed efficient inhibition of g-secretase activity demonstrated by a clear accumulation of APP C-terminal fragments (APP-CTFs), the direct substrate of PSEN1/g-secretase (data not shown). The dKO MEFs were then incubated overnight with N—[N-(3,5-difluorophenacetyl)-L-alanyl]-S-phenylglycine t-butyl ester (DAPT). DAPT is a non-transition state analogue (TSA) g-secretase inhibitor that is widely used (Dovey et al 2001 J Neurochem 76). Unexpectedly, upon DAPT treatment (1 μM, 16 hrs), EGFP-PSEN1 changed from a broad distribution towards a more restricted cytoplasmic localization co-enriching in LAMP1-positive organelles, hence resembling PSEN2 localization (FIG. 1A). Quantification using Mander's coefficient showed a significant threefold increase of EGFP-PSEN1 in these organelles (FIG. 1B). As PSEN2 is restricted to LAMP1-positive organelles (Sannerud et al 2016 Cell 166), no additional effect of DAPT was noticed here (FIG. 1A-B). Also, when quantifying the ratio of EGFP-PSEN1 fluorescence within the LAMP1-positive compartments over total EGFP-PSEN1, a significant 50% increase was noticed upon DAPT treatment, while the ratio for EGFP-PSEN2 remained unaltered (FIG. 1C). DAPT treatment had no measurable effect on the total size area of LAMP1-positive organelles (FIG. 1D) nor the steady state of PSEN1 or PSEN2 protein levels (data not shown), indicating that 1 μM DAPT did not affect the degradative endosomal route nor g-secretase turnover.

Next, we knocked out nicastrin (NCT) in our PSEN dKO MEFs using Crispr/Cas9 and subsequently stably rescued these triple KO MEFs with EGFP-PSEN1 and NCT C-terminally fused to a SNAP-tag (NCT-SNAP). It was confirmed that both tagged subunits integrated in functional and active g-secretase complexes by the stabilization of endogenous PEN2 levels, endoproteolysis of EGFP-PSEN1 and the normalized processing of APP-CTF (data not shown). Subsequent confocal analysis of DAPT-treated MEFs demonstrated that EGFP-PSEN1 and NCT-SNAP co-accumulate in LAMP1-positive organelles conclusively demonstrating the relocation of g-secretase complexes and not single EGFP-PSEN1 molecules (FIG. 1E). Identical observations were made in DAPT-treated single PSEN1 KO MEFs stably transduced with EGFP-PSEN1 (data not shown) and rat primary hippocampal neurons lentivirally transduced with GFP-PSEN1 (FIG. 1F), underscoring a conserved mechanism selective for PSEN1/g-secretase and not influenced by PSEN2 expression.

As LAMP1 immunoreactivity identifies both late endosomes and lysosomes, we next probed for a more specific marker for late endosomes, i.e. lysobisphosphatidic acid (LBPA) (Kobayashi et al 1998 Nature 392; Kobayashi et al 2002 J Biol Chem 277). EGFP-PSEN1 even more strongly co-distributed with anti-LBPA immunoreactivity identifying the late endosomal compartment as the final destination of DAPT-induced redistributed PSEN1/g-secretase (FIG. 1G). This was further corroborated by correlative light and EM (CLEM) using Lyso-Sir as a marker to identify EGFP-PSEN1 positive organelles (data not shown). We next wondered whether the observed relocation effect was selective for DAPT or a general feature of g-secretase inhibitors. We therefore tested, besides DAPT, two other inhibitors: Semagacestat (or LY450139), a DAPT-related non-TSA (allosteric) inhibitor (Siemers et al 2005 Clin Neuropharmacol 28; Tagami et al 2017 Cell Rep 21) and the TSA inhibitor, L-685-458 (also called Inhibitor X) (Shearman et al 2000 Biochemistry 39) (IC₅₀˜17±8 nM). For each inhibitor, we tested a dose response (1, 10, 100 and 1000 nM) for the mislocalisation of PSEN1/g-secretase in LAMP1-positive organelles. All three inhibitors re-located PSEN1/g-secretase to the late endosomes (FIG. 2A-2D). Interestingly, while we observed a clear dose response for DAPT and Semagacestat between 1 and 1000 nM, L-685-458 was already plateauing at 1 nM. Further lowering the concentration decreased the PSEN1/g-secretase accumulation in LAMP1-positive organelles dose-dependently, indicating a higher sensitivity for L-685-458 in re-locating PSEN1/g-secretase. On the other hand, the fold increase by which PSEN1/g-secretase relocates was almost two-fold for DAPT and Semagacestat, while less (1.5-fold) for L-685-458.

### Example 2. The g-Secretase Inhibitor-Induced Re-Location of PSEN1/g-Secretase to Late Endosomes is Reversible

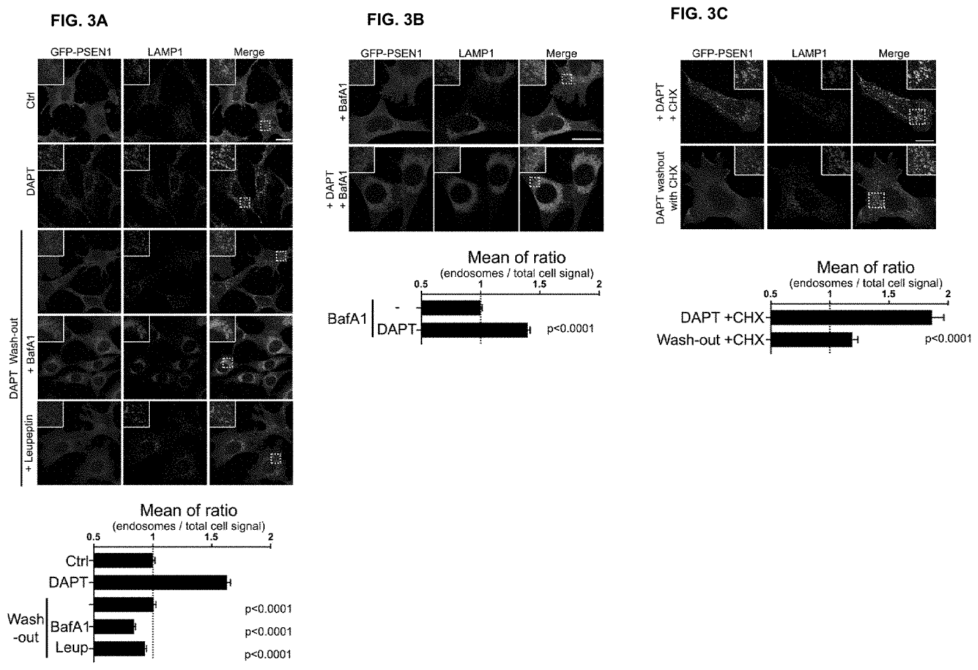

The g-secretase inhibitor-induced late endosomal localization of PSEN1/g-secretase made us wonder whether g-secretase was relocated there for lysosomal degradation. To test this, we first performed a wash-out experiment by simply replacing the DAPT-containing medium with fresh medium without inhibitor. After 16 hrs, EGFP-PSEN1 was found re-distributed and no longer accumulated in LAMP1-positive organelles indicating that the g-secretase inhibitor effect is fully reversible (FIG. 3A). Moreover, blocking lysosomal degradation or acidification during the wash-out period by adding Leupeptin or BafilomycinA1 (BafA1) respectively, did not prevent a normalized redistribution. This indicates that the re-located PSEN1/g-secretase in late endosomes is not a pool of proteins destined for degradation. In support, treating MEFs with BafA1 alone did not result in PSEN1/g-secretase accumulation in late endosomes, unless DAPT was added (FIG. 3B), suggesting that merely blocking lysosomal acidification (and thus functions including fusion and degradation) is not triggering redistribution. Finally, including cycloheximide (CHX) during the DAPT treatment did also not prevent PSEN1/g-secretase from redistributing to late endosomes (FIG. 3C). This rules out that the g-secretase inhibitor-induced accumulations originate from de novo synthetized PSEN1/g-secretase. Together, these results show that the translocation of PSEN1/g-secretase upon a treatment with g-secretase inhibitors is reversible and does not involve a de novo or degradable pool of complexes.

### Example 3. Administration of g-Secretase Inhibitors Primarily Affects Translocation of PSEN1/g-Secretase

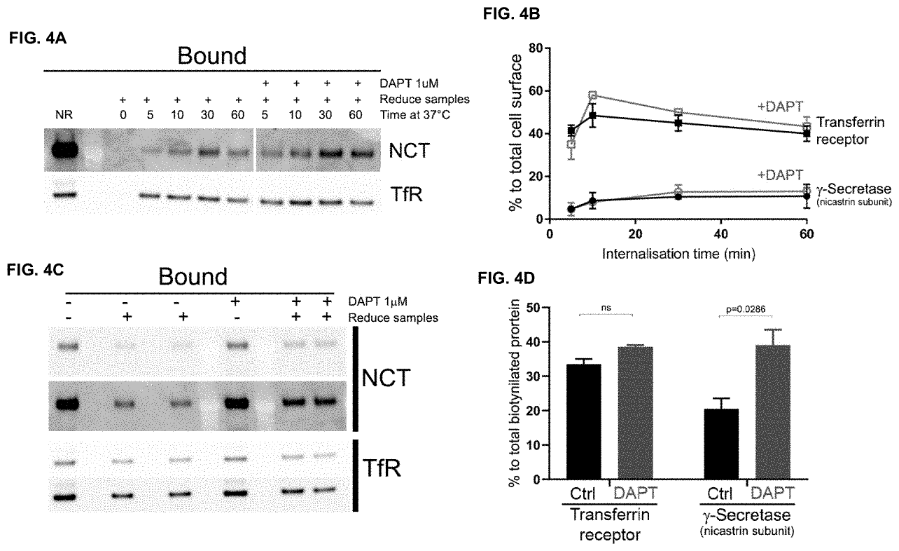

Our data so far indicate that the pool of PSEN1/g-secretase accumulating in late endosomes upon g-secretase inhibitor treatment likely originates from a derailed transport regulation in endosomal compartments. To test this, we first analyzed the effect of DAPT on internalization kinetics of PSEN1/g-secretase complexes focusing on NCT (as this subunit is most efficiently biotinylated) and using the transferrin receptor as a positive control. Cell surface biotinylation using a cleavable biotin analogue did not reveal significant differences in internalized NCT (FIG. 4A, quantified in 4B). However, at a 16 h time point, we observed a doubling of the amount of biotinylated NCT being pulled down following DAPT treatment, while no differences were found for the transferrin receptor (FIG. 4C, quantified in 4D). As all biotinylated proteins that are recycling to the cell surface are stripped from biotin prior to lysis and pull down, these data clearly suggest that a larger fraction of biotinylated NCT remained internalised upon DAPT treatment. As internalization kinetics were not altered, these data clearly point to a defect or delay in endosomal recycling of PSEN1/g-secretase. Furthermore, and based on the late endosomal accumulation, complexes are either re-routed from recycling to late endosomes, or, alternatively, recycling directly from late endosomes is affected by DAPT.

### Example 4. Translocated PSEN1/g-Secretase is Less Sensitive for g-Secretase Inhibition

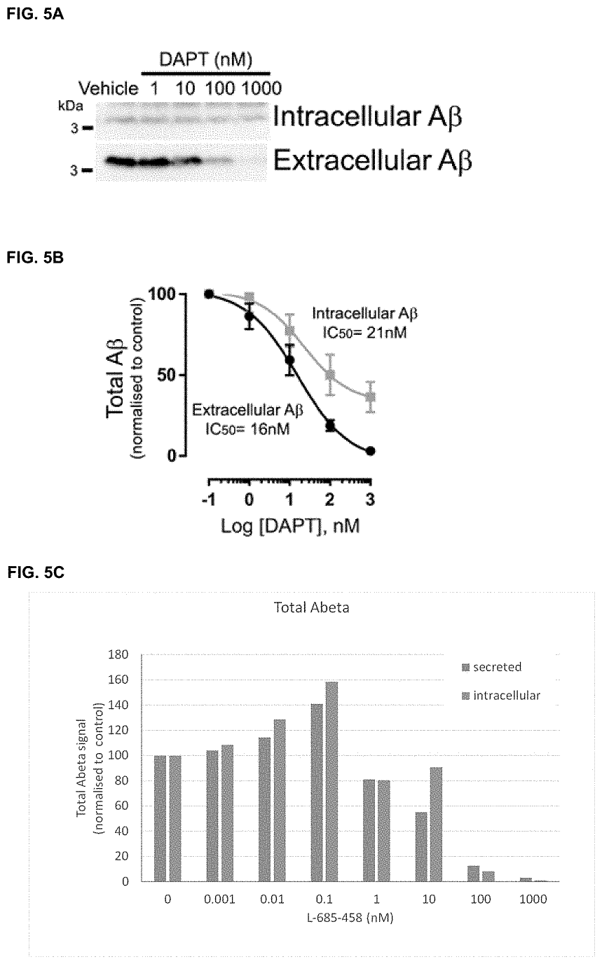

Over the years, g-secretase inhibitors have been developed for the sole reason to inhibit the production of amyloidogenic Ab fragments, hence blocking the further development of Ab plaques in AD patients' brains. In current application it is demonstrated for the first time that these g-secretase inhibitors translocate the PSEN1/g-secretase to the late endosomes, a subcellular compartment where g-secretase complexes preferentially produce those amyloidogenic Ab fragments. Based on these new data, it is crucial to compare the sensitivity to g-secretase inhibitors of the g-secretase complex located in the late endosomes (producing intracellular Ab) and the broadly localised g-secretase complex (producing extracellular Ab). We therefore compared the dose-dependencies of translocated PSEN1/g-secretase with catalytic inhibition by DAPT. MEFs were transfected with APP-C99-3×FLAG for 6 hours and treated with different doses of DAPT. After 16 hours, conditioned media and cell lysates were collected and analysed by Western blotting for total secreted and intracellular Ab, respectively. This resulted for DAPT in a lower IC50 for secreted Ab inhibition (FIG. 5A-B). Intracellular Ab had a higher IC50 (21 nM vs 16 nM) resulting in still higher Ab levels at the highest dose (1000 nM). The results described in current application are in line with the observation that the total Ab production upon administration of g-secretase inhibitors (e.g. L-685-458 see FIG. 5C) is increased in the low dose ranges and that the intracellular Ab production is higher than the secreted Ab production (FIG. 5C).

### Example 5. G-Secretase Stabilizers do not Affect PSEN1/g-Secretase Localisation

G-secretase inhibitors as those described above, inhibit not only the production of (toxic or longer) Ab peptides, but indifferently inhibit intramembrane proteolysis of other substrates leading to harmful side effects, as evidenced for instance through blocking the Notch signalling pathway (De Strooper and Annaert 2010 Annu Rev Cell Dev Biol 26). Therefore, alternatives for g-secretase inhibitors have been proposed. Small molecules, i.e. g-secretase stabilizers, have been developed to increase g-secretase processivity thereby promoting the generation of shorter Ab peptides (Szaruga et al 2017 Cell 170). We questioned whether those stabilizers would translocate the PSEN1/g-secretase in the same way as the inhibitors. The stabilizer GSM-4 (Kounnas et al 2010 Neuron 67, 769-780) was tested in our assay. Interestingly, GSM-4 did not induce a relocation of PSEN1/g-secretase to late endosomes in the dose range examined (FIG. 2E). Only a high non-physiological concentration of 10 μM resulted in some re-routing, but this is far beyond the IC50 of Ab inhibition reported for this modulator (IC50=131 nM).

### Example 6. FAD-PSEN1 Mutations Also Affect PSEN1/g-Secretase Localisation

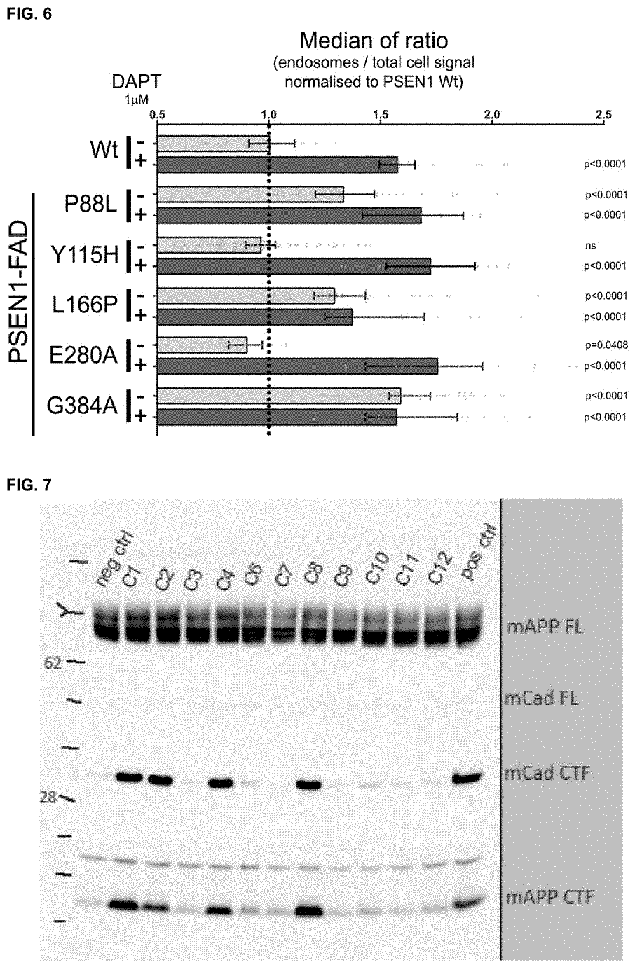

Previously Sannerud et al (2016 Cell 166) demonstrated that the FAD-associated mutations PSEN1-L166P and PSEN1-G384A on their own shifted PSEN1/g-secretase to late endosomes. As these mutations were reported to have an early onset and high thermo-instability (reflecting lowest processivity), we extended this experiment to other mutations reported in Szaruga et al (2017 Cell 170). In line with the above, the PSEN1-P88L mutation translocated PSEN1/g-secretase to late endosomes as well. Treatment of these mutant PSEN1 expressing MEFs with DAPT did not further increase late endosomal localization (FIG. 6). Two other mutations Y115H and E280A did not shift PSEN1/g-secretase to late endosomes, unless treated with DAPT. These mutations have a higher onset and affect the Ab processivity less as compared to L166P, G384A and P88L mutants.

### Example 7. Confirmation of the g-Secretase Inhibitor-Induced Translocation of PSEN1/g-Secretase to Late Endosomes

The obtained results described in Examples 1 and 5 (FIG. 2B-E) are very clear and convincing. However, to further confirm that the translocation effect of g-secretase inhibitors is not a stand-alone observation for some specific inhibitors but instead is a general characteristic for all g-secretase inhibitors, 4 additional inhibitors were tested: Avagacestat (referred to herein as C1), MK-0752 (referred to herein as C2), Begacestat (referred to herein as C4) and ELND006 (referred to herein as C8).

Avagacestat (BMS-708163; MolPort-009-679-474) is a highly potent g-secretase inhibitor (Aβ40 IC50=0.3 nM). It exhibits >137-fold selectivity for amyloid-beta precursor protein (APP) processing over Notch. It was previously shown to reduce plasma and brain Ab40 and Ab42 levels in vivo (Gillman et al 2010 ACS Med Chem Lett 1: 120 PMID: 24900185; Albright et al 2013 J Pharmacol Exp Ther 344: 686 PMID: 23275065). MK-0752 (MolPort-023-293-504) is a moderately potent g-secretase inhibitor (Aβ40 IC50=5 nM). MK-0752 both blocks Notch-intracellular domain (ICD) cleavage and its subsequent nuclear translocation as reduces the generation of Ab40 in plasma, brain and cerebrospinal fluid (Cook et al 2010 J Neurosci 30: 6743-6750; Harrison et al 2010 Cancer Res 70: 709-718). Begacestat (MolPort-023-277-132) is a selective thiophene sulfonamide inhibitor of g-secretase (Aβ40 IC50=15 nM). Begacestat treatment (5 mg/kg, p.o. in mice) for 4 h was shown to significantly reduce the brain Ab40 and Ab42 (Mayer et al 2008 J Med Chem 51:7348-7351). ELND006 (MFCD28386354) is an allosteric g-secretase inhibitor that selectively blocks Ab over Notch production both in cell-free (Ab IC50/Notch IC50=0.34 nM/6.6 nM) and cell-based assays (Ab IC50/Notch IC50=1.3 nM/85 nM).

In parallel, 7 different g-secretase stabilizers were tested: Flurizan (referred to herein as C3), TC-E 5006 (referred to herein as C6), Sulindac sulfide (referred to herein as C7), CHF 5074 (referred to herein as C9), E-2012 (referred to herein as C10), NGP-555 (referred to herein as C11) and GSM1 (referred to herein as C12).

Flurizan (MolPort-003-936-370) is a nonsteroidal anti-inflammatory drug that selectively lowers Ab42 levels in human neuroglioma cells (Eriksen et al 2003 J Clin Invest 112:440 PMID: 12897211). TC-E 5006 or Racemic (MolPort-035-765-874) reduces Ab42 levels in vitro (Ab42 IC50=390 nM) and in vivo (Peng et al 2011 ACS Med Chem Lett 2:786 PMID: 24900267). Sulindac sulfide (MFCD00869764) is a cell-permeable, active metabolite of sulindac (Cas nr 38194-50-2) and inhibits the secretion of Ab42 in CHO cells stably transfected with both APP75, and the PS1 mutant M146L. CHF5074 (1-(3′,4′-Dichloro-2-fluoro[1,1′-biphenyl]-4-yl)-cyclopropanecarboxylic acid or Itanapraced, CSP-1103) reduces Ab42 and Ab40 secretion, with an IC50 of 3.6 and 18.4 μM, respectively (Imbimbo et al 2007 J Pharmacol Exp Ther 323(3):822-830). E-2012 (CS-0293) is a potent g-secretase modulator without affecting Notch processing (Nakano-Ito et al 2014 Toxicol Sci 137(1):249-258). NGP-555 (CS-0030521) potently lowers Ab42 in vitro and in vivo while increasing shorter forms of Ab. Kounnas et al 2017 Alzheimers Dement 3(1):65-73). GSM1 is known as g-secretase modulator 1 (WO2009087127A1).

First, in the PSEN1 KO mouse embryonic fibroblasts (MEFs) rescued with EGFP-PSEN1 (see Example 1) the expression of the g-secretase subunits was tested and found not affected by administering any of the 12 compounds, including DAPT that was used as positive control (data not shown).

Second, of all these compounds, the inhibitory action on g-secretase activity was determined by quantifying the production of the C-terminal fragments (CTF) of Cadherin and APP. Both Cadherin-CTF and APP-CTF accumulate when the enzymatic activity of the g-secretase complex is inhibited. As can be appreciated from the Western blot in FIG. 7, all tested g-secretase inhibitors (C1, C2, C4 and C8) induced the level of Cadherin CTF and APP CTF in contrast to the negative control and the g-secretase stabilizers.

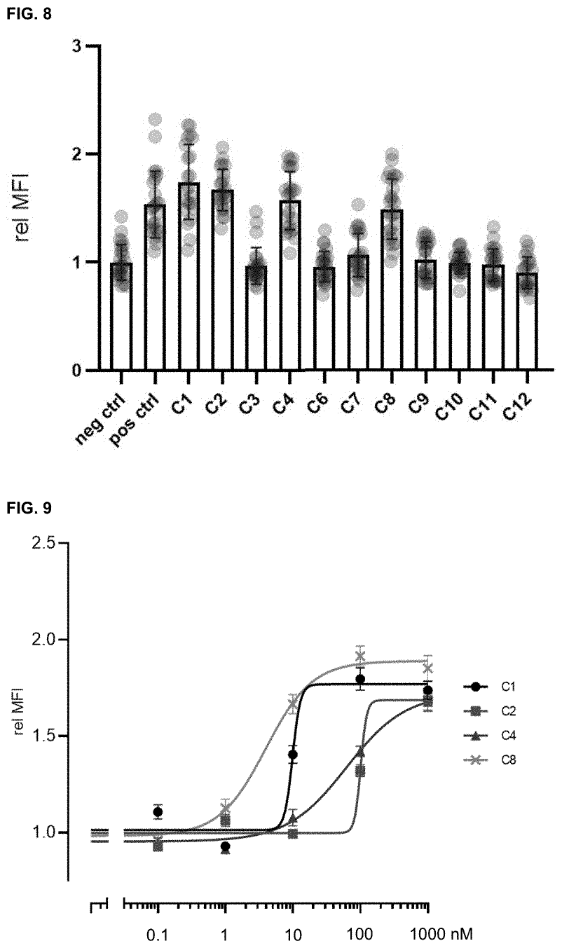

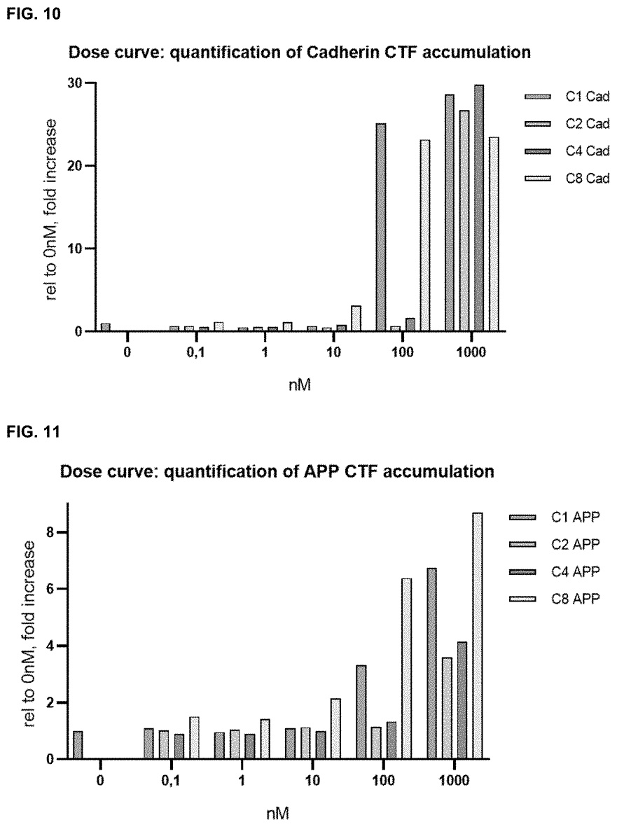

Third, the intracellular localisation of the PSEN1/g-secretase complex was quantified before and after administration of the compounds (FIG. 8). Fully in line with the results illustrated in FIG. 2B-E, all g-secretase inhibitors induced a translocation of the PSEN1/g-secretase complex to the late endosomes to a similar extent as the positive control DAPT, while none of the stabilizers did (FIG. 8). Interestingly, the dose-response curves of C1, C2, C4 and C8 on the translocation effect (FIG. 9) and on the Cadherin-CTF and APP-CTF accumulation (FIG. 10-11) confirm that g-secretase inhibitors already induce the translocation of the PSEN1/g-secretase complex towards the late endosome at concentrations that do not inhibit the enzymatic activity of the g-secretase complex yet.

Experimental Procedures

Cell Culture, Generation of Stable Cell Lines and Primary Hippocampal Neuron Culture

All culture media and sera are from ThermoFisher Scientific. Mouse embryonic fibroblasts (MEFs), PSEN1 knock-out (1 ko), PSEN1 and PSEN2 double knock-out (dKO, Herreman et al 2000 Nat Cell Biol 2), rescued 1 ko and dKO cell lines cells were routinely grown in Dulbecco's modified Eagle's medium (DMEM/F12) supplemented with 10% fetal calf serum (FCS). All cell lines were maintained in a humidified chamber with 5% CO₂ at 37° C. Primary hippocampal neuron cultures are derived from E18 rat embryos (Esselens et al 2004 J Cell Biol 166). Stable rescued 1 ko and dKO cell lines were generated using replication-defective recombinant retroviral system (pMCSV vector, Clontech) expressing the gene of interest.

Antibodies and Chemicals

The following monoclonal antibodies were commercially obtained: rat anti-LAMP1 (sc-19992, Santa Cruz); mouse anti-transferrin receptor (TfR, clone H68.4, Life Technologies), mouse anti-Abeta (clone 82E1, IBL America). The following rabbit antibodies were purchased: rabbit anti-PSEN1 (ab24748, abcam), rabbit anti-PSEN2 (ab51249, abcam), rabbit anti-Pen2 (ab18189, abcam), goat anti-GFP (for Western blot, 600-101-215, Rockland), chicken anti-GFP (for immunofluorescence, GFP-1020, ayes Labs). Rabbit pAb against APP C-terminus (B63.3), and mAb against NCT (9C3) were generated in-house (Annaert et al 1999 J Cell Biol 147; Esselens et al 2004 J Cell Biol 166; Herreman et al 2003 Nat Cell Biol 2). DAPT ((2S)—N-[(3,5-Difluorophenyl)acetyl]-L-alanyl-2-phenyl]glycine 1,1-dimethylethyl ester, Tocris), L-685,458 (InSolution InhibitorX, Merck), Semagacestat (Selleck Chemicals), GSM4 (corresponds to GSM(B) from Szaruga et al 2017 Cell 170), BafilomycinA1 (bioaustralis, used at 100 nM), Cycloheximide (CHX, sigma, used at 30 μg/ml), SNAP-Cell 647-SiR (new England Biolabs), Avagacestat (C1) (R&Dsyster, Cat No. 6363), Begacestat (C4) (MCE, HY-14175), ELND006 (C8) (Sigma, SML2122), Flurizan (C3) (Tocris, Cat No. 4495), MK-0752 (C2) (MCE, HY-10974), sulindac sulfide (C7) (Sigma, S3131), BMS-698861 (Glixx labs, PC-62912), CHF5074 (C9) (MCE, HY-14399), E-2012 (C10) (MCE, HY-10016), GSM1 (C12) (MCE, HY-10043), NGP-555 (C11) (MCE, HY-108714), TC-E5006 (C6) (Tocris, Cat No. 4898).

Plasmids, Transfection

The plasmids pSG5-C99-3×FLAG and pMSCV-GFP-PSEN1-wt and FADs, pFUGW-GFP-PSEN1 were previously described (Sannerud et al 2016 Cell 166). Mutations in pMSCV-GFP-PSEN2-AxxxAA were introduced using Q5 Site-Directed Mutagenesis Kit (New England BioLabs) according to the manufacturer with the forward primers: P94L: 5′-CTGTTTGTGCTTGTCACTCTG-3′; L172P: 5′-CCATGGCTGGCCGATCATGTCTTC-3′; G365A 5′-CTTGGCCTCGCGGACTTCATC-3′; and their reverse complement. Cells were transfected with FugeneHD (Roche Diagnostics) to express C99-3×FLAG. Primary hippocampal neurons were infected at 3 to 4 days in vitro (DIV) and analyzed at 7 DIV.

Virus Production

For virus production, HEK293T cells were transiently transfected using FuGENE6 (Promega) according to the manufacturer instruction. For retrovirus packaging, pMSCV expressing the gene of interest was co-transfected with the helper plasmid plk (Ecopac). Lentiviral particles were produced by co-transfection of appropriate lentiviral vectors with packaging (pCMV-ΔR8.74) and envelope (pMD2.G) plasmids in HEK293T cells. Lentiviral Particles were purified by ultracentrifugation and resuspended in DMEM/F12 medium. For transduction, viral particles were diluted in medium containing Polybrene (8 ng/μ1, Sigma). After 24 h, medium was refreshed. To establish stable cell lines, transduced cells were selected using 3 μg/ml puromycin (Sigma).

Western Blot

Protein concentrations were determined by the Bio-Rad DC protein assay (Bio-Rad). Samples were separated by SDS-PAGE (4-12% Bis-Tris NuPAGE gels in MES running buffer (ThermoFisher Scientific) and transferred onto nitrocellulose membranes (ThermoFisher Scientific). After blocking in 5% non-fat milk, membranes were incubated with primary antibody (4° C., overnight) followed by washing and incubation with horseradish peroxidase (HRP)-conjugated secondary antibodies (1 h, room temperature). After final washing, immunodetection was done using enhanced chemiluminescence (Western Lightning-Plus ECL, PerkinElmer), and immunoreactive protein bands were digitally captured and quantified on a Fuji MiniLAS 3000 imager (Fuji, Dusseldorf, Germany) using Aida Image Analyzer software (Raytest, Germany). Data are represented as mean±SEM of at least three independent experiments.

Confocal Laser Scanning Microscopy and Quantification

Cells were plated on glass coverslips, transfected 24 h later and processed for indirect immunolabeling the next day (Sannerud et al 2011 Proc Natl Acad Sci USA 108). Images were captured on a confocal microscope (Leica TCS SP5 II, Leica Microsystems) connected to an upright microscope, using an oil-immersion plan Apo 60× A/1.40 NA objective lens. Image acquisition was performed with LAS (Leica Microsystems). Images were further processed with ImageJ and PhotoshopCS6 (Adobe, Calif.). ImageJ software with the plugin JACoP (Bolte and Cordelieres 2006 J Microsc 224) was used for signal overlap quantification (Mander's coefficient). Images were acquired with identical settings and regions of interest (ROI) covering whole cells were used for quantification.

Cell Surface Biotinylation Internalization Assay

All reagents, except otherwise stated, were kept on ice. Cells (450.000 cells for 1 h kinetic and 300.000 cells for 16 h) were plated on 6-cm dishes. 48 h after plating, cells were placed on ice, washed in PBS (pH 8) and next incubated in PBS (pH 8) supplemented with 0.25 mg/ml sulfo-NHS-SS-Biotin (Pierce) (10 min at 4° C.). Excess of biotin was washed out with PBS and cells were incubated in quench buffer (0.5% BSA, 100 mM Glycine in PBS) for 15 min at 4° C. Cells were then washed with PBS and incubated at 37° C. in complete pre-warmed medium (with or without DAPT 1 μM) for the appropriate time or kept at 4° C. as a control. Endocytosis was quickly stopped by placing the dishes back on ice and washing the cells with ice-cold PBS. Remaining biotin at the cell surface was cleaved off by incubating the cells twice in PBS containing 100 mM 2-sodium-2-mercaptoethanesulfonate (Sigma) as a reducing agent (15 min at 4° C.). To determine total surface biotinylation cells were incubated in PBS lacking the reducing agent. After this, reduced cells were washed with ice-cold 5 mg/ml iodoacetamide for 5 min, then twice with ice-cold PBS, and finally extracted in lysis buffer (50 mM HEPES, pH 7.2, 100 mM NaCl, 1% Triton X-100, with proteases inhibitor cocktail, Sigma). Total protein was measured and biotinylated proteins were pulled down from equal amounts of extracts using streptavidin Sepharose beads (Pierce) (4° C., overnight on a rotation wheel). After washing, the bound material was eluted from the beads using 2× loading buffer (Invitrogen) containing 2% beta-mercaptoethanol (70° C. for 10 min), separated on precasted 4-12% Bolt gels in MES running buffer (Invitrogen) and processed for Western blotting and immunodetection. For each experiment, two control samples were prepared, one being non-reduced (NR) but kept at 4° C. during the whole procedure (hence representing the total pool of surface biotinylated proteins), one reduced (R0) but kept at 4° C. (to monitor the efficiency of reduction of biotin). To evaluate the internalized pool for each protein to total cell surface, the (R37-R0)/NR*100 ratio for each time point were quantified. For the 16 h time point, the R37/NR*100 were quantified for each condition. Each experiment was performed two or three times. Values are presented as mean±SEM.

Graphical Representations and Statistical Analysis

Graphical representations and statistical analyses were performed with Prism 8.0 (Graph Pad Software, La Jolla, Calif., USA). Quantification are represented by bar graph (median or mean as indicated) including individual data points. Two data sets were compared using Mann-Whitney rank test (two-sided). Dose response curves were fitted with variable slope (four parameters)

Generating Knockout Cells Using CRISPR/Cas9

The web-based CRISPR Design Tool (http://crispor.tefor.net/) was used to select the genomic sequence target in mouse PSEN1 (5′-caacgttatcaagtacctccccgaa-3′), PSEN2 (5′-caacgtcctgggcgaccgtcgggcc-3′) and NCT (5′-caccgcgttgagcaggcggacaca-3′). Oligo pairs (Integrated DNA Technologies) encoding guide sequences were annealed and ligated into the plasmid pX330 (Addgene) for PSEN1 and PSEN2 and px459 for NCT following Zhang's laboratory protocol (https://www.addgene.org/crispr/zhang/). To generate PSEN1/PSEN2 double knock out (PSdKO), MEF cells were co-transfected with pX330-PSEN1 and pX330-PSEN2 using FugeneHD (Promega). Selection of dKO clones was done by serial dilution followed by Western blot analysis. Once PSdKO clones were selected, cells were transfected with px459-NCT vector followed by three days selection with puromycin (3 mg/ml) to select for transfected cells. Then cells were proliferated and selection of NCT KO clones was done by serial dilution followed by Western blot analysis.

1. A method of screening a gamma-secretase inhibitor for inhibition of Amyloid beta (Ab) fragment production, the method comprising:
determining the cellular localisation of a gamma-secretase complex comprising presenilin1 in cells of a cell culture that express the gamma-secretase complex;
administering the gamma-secretase inhibitor to the cell culture;
determining the cellular localisation of the gamma-secretase complex in the cells of the cell culture and after administration of the gamma-secretase inhibitor;
determining that the gamma-secretase complex is at most 50% more translocated to the late endosomes after the administration of the gamma-secretase inhibitor compared to before the administration.

2. A method of screening for an inhibitor of amyloidogenic Amyloid beta (Ab) fragment production, the method comprising:
providing a cell culture wherein cells in the cell culture express a gamma-secretase complex comprising presenilin1;
determining the cellular localisation of the gamma-secretase complex in the cell culture and quantifying Ab peptides with a length of 38, 40, 42 and/or 43 amino acids in the cell culture prior to the administration of a test compound;
administering to the cell culture the test compound;
determining the cellular localisation of the gamma-secretase complex in the cell culture and quantifying Ab peptides with a length of 38, 40, 42 and/or 43 amino acids in the cell culture after to the administration of the test compound;
determining that the gamma-secretase complex is at most 50% more translocated to the late endosomes after administration of the test compound compared to before the administration of the test compound and that there is a statistically significant increase in the ratio of Ab38/Ab42, Ab40/Ab42, Ab40/Ab43 or of Ab(38+40)/Ab(42+43) after administration of the test compound compared to before the administration of the test compound.

3. The method according to claim 2, wherein Ab peptides are quantified via immune-based assays.

4. The method according to claim 1, wherein the localisation of the gamma-secretase complex is determined by the localisation of presenilin1.

5. The method according to claim 1, wherein the location of the gamma-secretase complex is measured by the ratio of presenilin1 level in late endosomes to the total cellular presenilin1 level.

6. The method according to claim 5, wherein presenilin1 is fluorescently labelled and wherein the presenilin1 level corresponds to the presenilin1 fluorescent signal.

7. The method according to claim 1, wherein the late endosomes are visualised by LAMP1 or by lysobisphosphatidic acid.

8. The method according to claim 1, wherein the cell culture consists of mammalian cells.

9. The method according to claim 1, wherein the gamma-secretase inhibitor is a therapeutic candidate for the prevention and/or treatment of Alzheimer's disease.

10. A mammalian cell devoid of endogenous nicastrin, presenilin1, and presenilin2 protein production, the cell comprising:
a labelled nicastrin, and
a labelled presenilin1 and/or presenilin2 protein.

11. The method according to claim 2, wherein the localisation of the gamma-secretase complex is determined by the localisation of presenilin1.

12. The method according to claim 2, wherein the location of the gamma-secretase complex is measured by the ratio of presenilin1 level in late endosomes to the total cellular presenilin1 level.

13. The method of claim 12, wherein presenilin1 is fluorescently labelled and wherein the presenilin1 level corresponds to the presenilin1 fluorescent signal.

14. The method according to claim 2, wherein the late endosomes are visualised by LAMP1 or by lysobisphosphatidic acid.

15. The method according to claim 2, wherein the cell culture consists of mammalian cells.

16. The method according to claim 2, wherein the inhibitor of amyloidogenic Ab fragment production is a therapeutic candidate for the prevention and/or treatment of Alzheimer's disease.


SEQUENCE LISTING (6 sequences)

SEQ ID NO: 1 - DNA, 21 bp, Artificial Sequence
  Primer
ctgtttgtgcttgtcactctg

SEQ ID NO: 2 - DNA, 24 bp, Artificial Sequence
  Primer
ccatggctggccgatcatgtcttc

SEQ ID NO: 3 - DNA, 21 bp, Artificial Sequence
  Primer
cttggcctcgcggacttcatc

SEQ ID NO: 4 - DNA, 25 bp, Mus musculus
caacgttatcaagtacctccccgaa

SEQ ID NO: 5 - DNA, 25 bp, Mus musculus
caacgtcctgggcgaccgtcgggcc

SEQ ID NO: 6 - DNA, 24 bp, Mus musculus
caccgcgttgagcaggcggacaca